(19) United States
(12) Patent Application Publication
(10) Pub. No.: US 2007/0059343 A1
(43) Pub. Date: Mar. 15, 2007

(54) Pet life

(76) Inventor: Sridat Surendrananth Chinsammy, Schenectady, NY (US)

Correspondence Address: SRIDAT CHINSAMMY, 429 BRANDY WINE AVE, SCHENECTADY, NY, 12308, US

(21) Appl. No.: 11/415,236

(22) Filed: Oct. 10, 2006

Related U.S. Application Data
(60) Provisional application No. 60/677,109, filed on May 3, 2005.

(51) Int. Cl. A61K 36/254; A23K 1/165

(52) U.S. Cl. 424/442; 424/728

(57) ABSTRACT

My idea/invention of “PetLife” is the formulation of unique chemical formulas for the production of fortified instant liquid energy drink for dogs and cats, in the individual and dual sense; as to say “Pet Life Dogs”, “Pet Life Cats” and “Pet Life General”. To boost there vitality and strength within a short period of time from consumption

Listed below are the linkages of ingredients can be used to produce two of the beverages associated to my idea/invention of “PetLife”, as described in the abstract of my provisional patent application (60/677,109), accompanied by there nutritional values. Considering the fact that the anatomy and metabolic reactions of both dogs and cats (mammals) of all species are similar to that of humans in many ways even though there is no audible effect, when synchronize under the very same chemicals exposure.

Capitalizing on the fact that it is commonly know that regularly researchers uses these species of animals to run trials to determine the effectiveness of special medications that are intended for humans. It is scientifically logical to conclude that these species can also consume the very same type of energy beverages that humans does and it will serves the very same purpose as intended in humans. The effectiveness of these beverages that my idea/invention purports is dependent on the potency of its functional ingredient/s.

(1.) Ingredients: Carbonated Water, L-Taurine, L-Glutamine, Citric Acid, Natural Flavors, Adaptogenic Blend (Elutherococcus Senticosus, Panax Ginseng, Panax Quinquefolium, Schisandra, Astragalus, and Reishi), Acesulfame Potassium, Caffeine, Sodium Benzoate, Potassium Sorbate Pantothenic Acid, Sucralose, Cyanocobalamin, Niacin, Pyrodoxine HCI, and Yellow 6. **Protein source from freeform amino acids.

(2.) .) Ingredients: Carbonated Water, Taurine, Cranberry Juice Concentrate, Citric Acid, Natural Flavors, Ascorbic Acid, Acesulfame Potassium, Sodium Benzoate, Potassium Sorbate Niacin, Pantothenic Acid, Pyrodoxine HCI, and Red 40 .Folic Acid and Cyanocobalamin.” **Protein source from freeform amino acids.

### Compostition of Premix

Niacin (as Niacinamide, USP)
Pantothenic Acid (as D-Calcium Pantothenate, USP)
Vitamin B12 (as Cyanocobalamin, USP)
Vitamin B6 (as Pyrodoxine HCI, USP)
L-Glutamine (FCC)
Caffeine, Anhydrous(USP) & (Guarana Extract, 10% Caffeine (Soluble)
Guarana Extract, 10% Caffeine (Soluble)
Panax Ginseng P.E.W.S.

Taurine

Nutrition Facts
% Daily VALUE

| | | |
|---|---|---|
| Total Fat | 0 GM | 0% |
| Cholesterol | 0 GM | 0% |
| Sodium | 24 MG | 1% |
| Potassium | 25 MG | <1% |
| Total Carbohydrate | 0 GM | 0% |
| Sugars | 0 GM | |
| Protein** | 2 GM | 0% |
| Vitamin A | | 0% |
| Vitamin C | | 0% |
| Calcium | | 0% |
| Iron | | 0% |
| Vitamin B3 | | 100% |
| Vitamin B5 | | 100% |
| Vitamin B6 | | 300% |
| Vitamin B12 | | 4700% |

1. I Sridat Chinsammy am claiming sole proprietary rights on the idea/invention of “PetLife”, since I am the first person to conceive and protect the said as is clearly being described in the contents of my patent application. In the widest prospective.
